(19) United States
(12) Patent Application Publication
(10) Pub. No.: US 2010/0331606 A1
(43) Pub. Date: Dec. 30, 2010

(54) RELAXATION SYSTEM

(75) Inventors: Kwok Fong WONG, Kwai Chung (HK); Pui Yi Ching, Kwai Chung (HK)

Correspondence Address: SUGHRUE MION, PLLC, 2100 PENNSYLVANIA AVENUE, N.W., SUITE 800, WASHINGTON, DC, 20037, US

(73) Assignee: Kwok Fong WONG, Kwai Chung (HK)

(21) Appl. No.: 12/582,244

(22) Filed: Oct. 20, 2009

(30) Foreign Application Priority Data
Jun. 26, 2009 (HK) 09105795.7

(51) Int. Cl. A61M 21/00; G06F 17/00; G06K 9/00

(52) U.S. Cl. 600/27; 700/94; 382/124

(57) ABSTRACT

The present invention provides a relaxation system comprising a processing unit, a sound playing unit for playing sounds, a light image emitting unit for emitting light, a smell releasing unit for releasing smell and a fingerprint scanning unit for collecting fingerprint information and transmitting the same to the processing unit, in which the latter four units are connected to the processing unit respectively, the processing unit is used to control the operation of the sound playing unit, the light image emitting unit and the smell releasing unit and perform a fingerprint identification. The system further comprises an environmental sensor unit connected to the processing unit and being used to collect environmental information to enable the processing unit to control the operation of the sound playing unit, the light image emitting unit and the smell releasing unit. The environmental sensor unit comprises a microphone for detecting noise signal in the environment.

### FIELD OF THE INVENTION

The present invention relates generally to the field of health care, and more particularly to a relaxation system for relieving the stresses and tension of a user.

### BACKGROUND OF THE INVENTION

Given the increasingly fierce social competition in current society, people face to a variety of stresses from many aspects of daily life. The stress of an individual generally originates from the surrounding environment and is a psychological response to the latter. Most of the stresses could be exactly perceived, but some of them could not and are always ignored.

A series of physiological and psychological disorders might be incurred if the stresses accumulate over time and could not be relieved. There are many methods for stress relief, such as chatting, traveling, sporting, abreaction, massage, etc. However, it is difficult for the modern people to use those methods to relieve stress due to the tense and busy life.

To enable them to relieve stress regardless of the busy life, more and more massage products have been developed, such as massage chairs. Such massage products could relieve stress to some extent. However, as the cause of stress is very complicated, a massage to human body could only relieve the stress from one aspect, thus its effect of stress relief is very limited.

### SUMMARY OF THE INVENTION

To solve the above-mentioned problems, the object of the present invention is to provide a relaxation system capable of relieving the stress and tension of a user.

The relaxation system of the present invention may comprise a processing unit, a sound playing unit, a light image emitting unit, a smell releasing unit and a fingerprint scanning unit, in which the sound playing unit, the light image emitting unit, the smell releasing unit and the fingerprint scanning unit are connected to the processing unit respectively, the sound playing unit is used to play sounds, the light image emitting unit is used to emit light, the smell releasing unit is used to release smell, the fingerprint scanning unit is used to collect fingerprint information and transmit the collected fingerprint information to the processing unit, the processing unit is used to control the operation of the sound playing unit, the light image emitting unit and the smell releasing unit and perform a fingerprint identification according to the fingerprint information collected by the fingerprint scanning unit, the system further comprises an environmental sensor unit, and the environmental sensor unit is connected to the processing unit and is used to collect environmental information for the processing unit to enable the processing unit to control the operation of the sound playing unit, the light image emitting unit and the smell releasing unit according to the collected environmental information, the environmental sensor unit comprises a microphone for detecting noise signal in the environment.

Preferably, the smell releasing unit may comprise at least one fragrance carrier, and a microprocessor for selecting one of the at least one fragrance carrier and controlling the operation of the selected fragrance carrier.

Preferably, each of the at least one fragrance carrier may comprise a container for containing a kind of fragrance liquid, such as perfume or essential oil, a cylinder extending upwards from an opening provided with the container and comprising a through hole, a absorption element provided within the through hole of the cylinder for conveying the fragrance liquid contained in the container, an electrical heating element attached to the cylinder and connected to a power source to be supplied with power. The cylinder could be made of metal or other thermal conductive materials.

Preferably, one or more of the at least one fragrance carrier could be associated with an anion generator to enable to produce anion during the operation of the former.

Preferably, the relaxation system of the present invention may further comprise a storage unit for storing sounds and fingerprint information, in which the storage unit is connected to the processing unit, and the processing unit is used to determine whether fingerprint information collected by the fingerprint scanning unit is authorized in accordance with the fingerprint information stored in the storage unit and control the sound playing unit to play the sounds stored in the storage unit.

Preferably, the relaxation system of the present invention may further comprise a far infrared ray emitting unit for emitting far infrared rays, wherein the far infrared ray emitting unit is connected to the processing unit, and the processing unit is used to control the far infrared ray emitting unit to emit far infrared rays.

Preferably, the environmental sensor unit of the present invention may comprise a heartbeat detector for detecting heartbeat rate of a user.

Preferably, the environmental sensor unit of the present invention may comprise a blood pressure meter for measuring blood pressure of a user.

Preferably, the light image emitting unit may be a light source capable of emitting light in a variety of colors, and be connected to one or more units among the sound playing unit, the smell releasing unit and the environmental sensor unit to enable to operate synchronously.

Preferably, the relaxation system of the present invention may further comprise a massage unit for offering a massage to a user.

The present invention may first identify an authorized user according to fingerprint thereof, and then stimulate the auditory, optical and olfactory nerve of the authorized user by virtue of the sound playing unit, the light image emitting unit and the smell releasing unit, to alter the brainwave pattern of the user, especially to smooth and balance the brainwave pattern of the user. Thus, the stress of the user could be relieved, and the incidence of the variety of psychological and physiological disorders resulting from the stress could be reduced.

### BRIEF DESCRIPTION OF THE DRAWINGS

To describe the embodiments of the present invention in details, the accompanying drawings illustrating those embodiments will be introduced as follows. Obviously, the accompanying drawings are only drawings for certain embodiments of the present invention. It should be understood that other drawings could be conceived by the skilled in the art according those accompanying drawings without creative work.

FIG. 1 is a block diagram of a relaxation system according to a first embodiment of the present invention;

FIG. 2 is a block diagram of a relaxation system according to a second embodiment of the present invention;

FIG. 3 is a block diagram of a relaxation system according to a third embodiment of the present invention;

FIG. 4 is a block diagram of another relaxation system according to the third embodiment of the present invention; and

FIG. 5 is a circuit diagram of a relaxation system according to a fourth embodiment of the present invention.

### DETAILED DESCRIPTION OF THE INVENTION

The objects, technical solutions, and advantages of the present invention will be more apparent from the following particular description of the preferred embodiments as illustrated in the accompanying drawings. The sample embodiments of the present invention and the description thereof are for illustration purposes only, and the present invention should not be limited to those embodiments.

### THE FIRST EMBODIMENT

Studies show that the stress of human being could be relieved though changing the brainwave pattern thereof. For example, the slower and smoother the brainwave is, the more relaxed and comfortable we will feel. Generally, the brain of human being generates 9-14 cycles of Alpha brainwaves per second when in a calm and buoyant emotional state, but would rise to 15-40 cycles per second during an exciting emotional state. Furthermore, the brainwave pattern of human being could be altered by virtue of the information perceivable by the sense of hearing, sight or smell thereof.

Therefore, the main object of the present invention is to provide a relaxation system which could alter the brainwave pattern of a tense and nervous user by stimulating his sense of hearing, sight or smell.

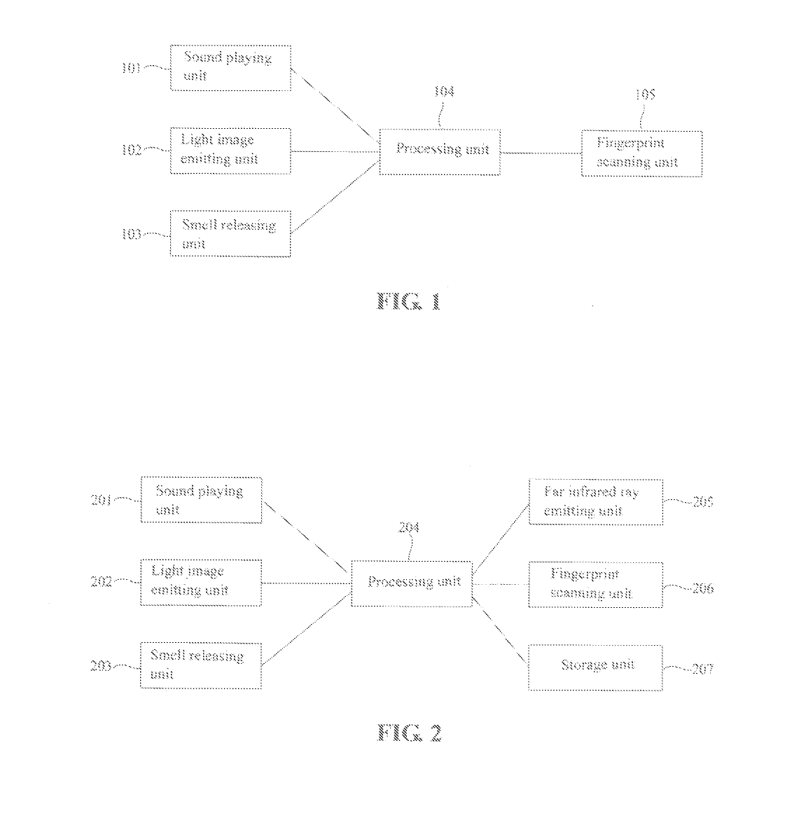

FIG. 1 illustrates a block diagram of a relaxation system according to the first embodiment of the present invention. The relaxation system comprises a sound playing unit 101, a light image emitting unit 102, a smell releasing unit 103, a processing unit 104, and a fingerprint scanning unit 105, in which the sound playing unit 101, the light image emitting unit 102, the smell releasing unit 103, and the fingerprint scanning unit 105 are connected to the processing unit 104 respectively.

The sound playing unit 101 is used to play sounds for stress relief, and comprises a speaker. Of course, the user could listen to the sounds by mean of insert earphones. In daily life, we may realize that some kinds of sounds could make us feel pleased and relaxed, while some might make us nervous. Since the auditory nerve of human being is connected with all the muscle through the inner ear (vibrosound memory), sounds could actuate the movement of human body. As a result, the stress of human being could be relieved or increased. This is because the brainwave pattern has been altered. Therefore, the sounds played by the sound playing unit 101 of this embodiment are sounds for relieving the stress of the user, and could be chosen according to the stress level of the user.

The light image emitting unit 102 is used to emit beams and/or images for stress relief. The beams and/or images emitted therefrom could stimulate the optic nerve of the user, thus, the brainwave pattern of the user could also be changed. Studies show that suitable beams and images, such as soft light, green light or green images, could relieve stress of human being. The light image emitting unit 102 of this embodiment could emit such kinds of images in accordance with the stress level of the user, to relieve the stress of the user. When the light image emitting unit 102 is used to emit light, it could be a light source capable of emitting light in a variety of colors. When the light image emitting unit 102 is used to emit images, it is in fact a device similar to a projector. Of course, the light image emitting unit 102 could be a combination of the above-mentioned two devices.

The smell releasing unit 103 is used to release smells for stress relief. The smells mentioned in this embodiment mainly refer to various fragrances. Studies show that suitable fragrance could stimulate the olfactory nerve of human being, thus the brainwave pattern of human being could also be altered, and then the stress could be relieved and the tense emotion could be relaxed. For example, the cool fragrance of lemon could make one feel excited and refresh from sleepiness, the fragrance of lavender could make one feel relaxed and eliminate his fatigue, while fragrance of rose could promote sleep. Obviously, the fragrances released from the smell releasing unit 103 of this embodiment could be applied to most of people rather than those who are allergic to or do not like some of the fragrances.

To release a corresponding smell according to the preference of a certain user, the smell releasing unit 103 may comprise at least one fragrance carrier and a microprocessor for selecting one of the at least one fragrance carrier and controlling the operation the selected fragrance carrier. Each of the fragrance carriers comprises a container for containing a kind of fragrance liquid, such as perfume or essential oil, a cylinder extending upwards from an opening provided with the container and comprising a through hole, a absorption element provided within the through hole of the cylinder for conveying the fragrance liquid contained in the container, an electrical heating element attached to the cylinder and connected to a power source to be supplied with power. The cylinder could be made of metal, such as iron, steel, copper, aluminum, or other thermal conductive materials.

When a fragrance carrier is selected by the microprocessor, the microprocessor would command the power source to supply power to the electrical heating element. Then, the electrical heating element will heat the cylinder and in turn heat the absorption element. The heated absorption element starts to absorb the fragrance liquid contained in the container and release the same to the ambient. Similarly, if another fragrance carrier is selected, the fragrance liquid contained in the container thereof would be released accordingly. For example, there are five fragrance carriers, one for fragrance of lemon, one for fragrance of lavender, one for fragrance of rose, one for fragrance of lily, while one for fragrance of mint. The microprocessor could select one of the five fragrance carriers to release the corresponding fragrance.

Furthermore, one of the fragrance carriers could be associated with an anion generator to form an air purifier. When the fragrance carrier associated with the anion generator is selected, it could not only release corresponding fragrance, but also produce a great deal of anion to purify air. The other fragrance carriers in the smell releasing unit could also be associated with the anion generator to cooperate with the latter.

The fingerprint scanning unit 105 is used for collecting fingerprint information of the user, and transmitting the collected fingerprint information to the processing unit 104. The processing unit 104 will compare the received fingerprint information against preset fingerprint information to determine whether the former corresponds with one piece of the latter, and then control the system to operate accordingly. In the embodiment, the user may preset a series of stress relieving measures in the relaxation system, and each of the measures corresponds to certain fingerprint information. After the processing unit 104 identifies a piece of fingerprint information by means of the above fingerprint identification, and retrieves the measure corresponding to the piece of fingerprint information, it will control the corresponding units of the system to perform the retrieved measure to relieve the stress of the user. For instance, the fingerprint information of Peter's index finger corresponds to a measure of playing the music named Hundreds Birds Worshipping The Phoenix by the sound playing unit 101, emitting soft green light by the light image emitting unit 102 and releasing the fragrance of rose by the smell releasing unit 103, while the fingerprint information of Mary's thumb corresponds to another measure of playing sea wave sound by the sound playing unit 101 and releasing the fragrance of lavender by the smell releasing unit 103.

As mentioned above, in order to relieve the stress of the user, the processing unit 104 is used to control the sound playing unit 101 to play the corresponding sound, the light image emitting unit 102 to emit the corresponding image and/or the smell releasing unit 103 to releasing the corresponding smell after determining that the fingerprint information of the user collected by the fingerprint scanning unit 105 corresponds with one of the preset fingerprint information through fingerprint identification. In practice, the processing unit 104 may be a microprocessor or a computer terminal in which the control program is preset.

The present embodiment identifies the user through fingerprint identification and stimulates the auditory, optic and/or olfactory nerve of the user by virtue of the sound playing unit, the light image emitting unit and the smell releasing unit, thus the brainwave pattern of the user could be altered. Particularly, the slow and smooth brainwave pattern could relieve the stress of the user, and could reduce the incidence of various physiological and psychological disorders resulted from the stress.

### THE SECOND EMBODIMENT

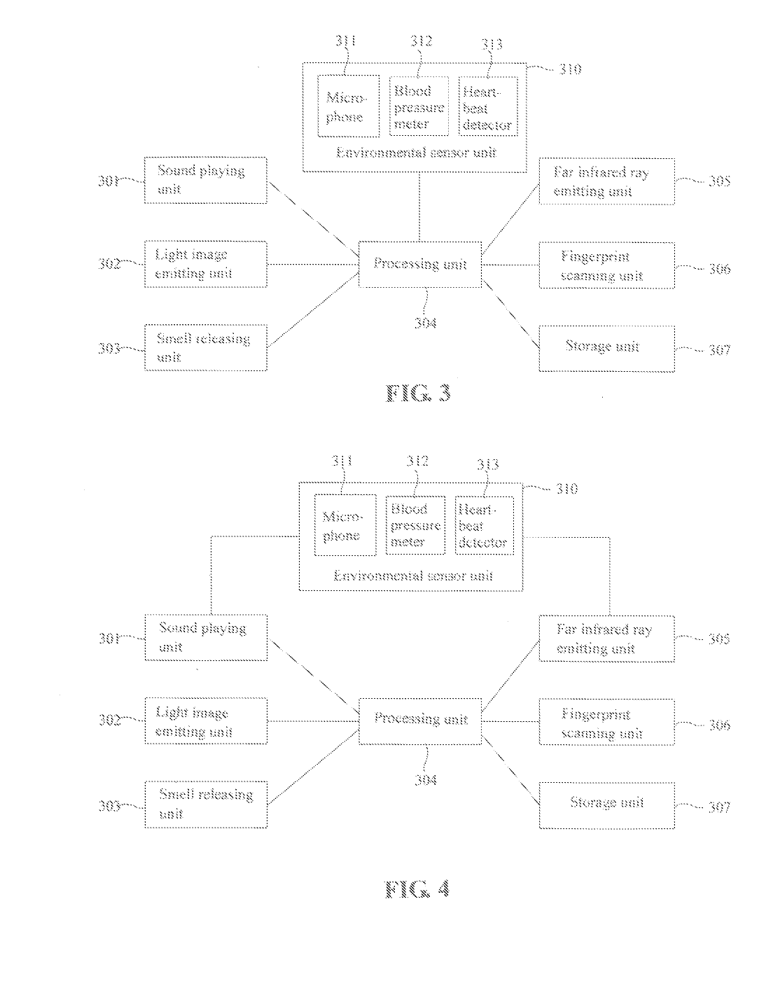

FIG. 2 illustrates a block diagram of a relaxation system according to the second embodiment of the present invention. The relaxation system comprises a sound playing unit 201, a light image emitting unit 202, a smell releasing unit 203, a processing unit 204, and a fingerprint scanning unit 206, in which the sound playing unit 201, the light image emitting unit 202, the smell releasing unit 203, and the fingerprint scanning unit 206 are connected to the processing unit 204 respectively. The structures of the above units are similar to those in the first embodiment, thus the details of which would not be repeated here.

The present embodiment further comprises a far infrared ray emitting unit 205 and a storage unit 207 both of which are also connected to the processing unit 204.

The far infrared ray emitting unit 205 is used to emit far infrared rays to relieve the stress of the user, which realize the function of stress relief by incurring various physical and chemical reactions with respect to the body of the user, rather than stimulating the senses of the user. The distinct advantage of the far infrared ray is that it is easy to penetrate the skin of the user and incur natural resonance with a plurality of beneficial effects, such as promoting blood circulation, managing blood pressure, skin care and beautifying, relieving joint pain, and so on. Since the far infrared ray has so many beneficial effects, it could delight the user by relieve his stress from many aspects.

The storage unit 207 is used to store the fingerprint information of the user or users in advance, as well as the sound information to be played by the sound playing unit 201. When the processing unit 204 needs to identify the fingerprint information collected by the fingerprint scanning unit 206, it could retrieve the preset fingerprint information. If the collected fingerprint information is identified, the processing unit 204 could control the sound playing unit 201 to play the corresponding sound stored in the storage unit 207.

As a sample of the present invention, at least two units among the sound playing units 201, the light image emitting unit 202, the smell releasing unit 203 and the far infrared ray emitting unit 205 could operate synchronously. For example, when the sound playing unit 201 plays sound, the light image emitting unit 202 would emit light image at the same time, while the far infrared ray emitting unit 205 would also emitting far infrared ray synchronously. The synchronization between two units could be realized by the control of the processing unit 204. However, it is also possible to realize the synchronization by connecting one of the two units to the processing unit 204 and then connecting the other one of the two units to the former unit. For instance, to make the sound playing unit 201 and light image emitting unit 202 keep pace with each other, the light image emitting unit 202 could be connected to the sound playing unit 201 directly. Thus, when the sound playing unit 201 plays sound under the control of the processing unit 204, it also actuate the light image emitting unit 202 to emit light image.

The present embodiment rapidly control the operation of the sound playing unit, the light image emitting unit, the smell releasing unit and the far infrared ray emitting unit to relieve the stress of the user by means of fingerprint identification.

### THE THIRD EMBODIMENT

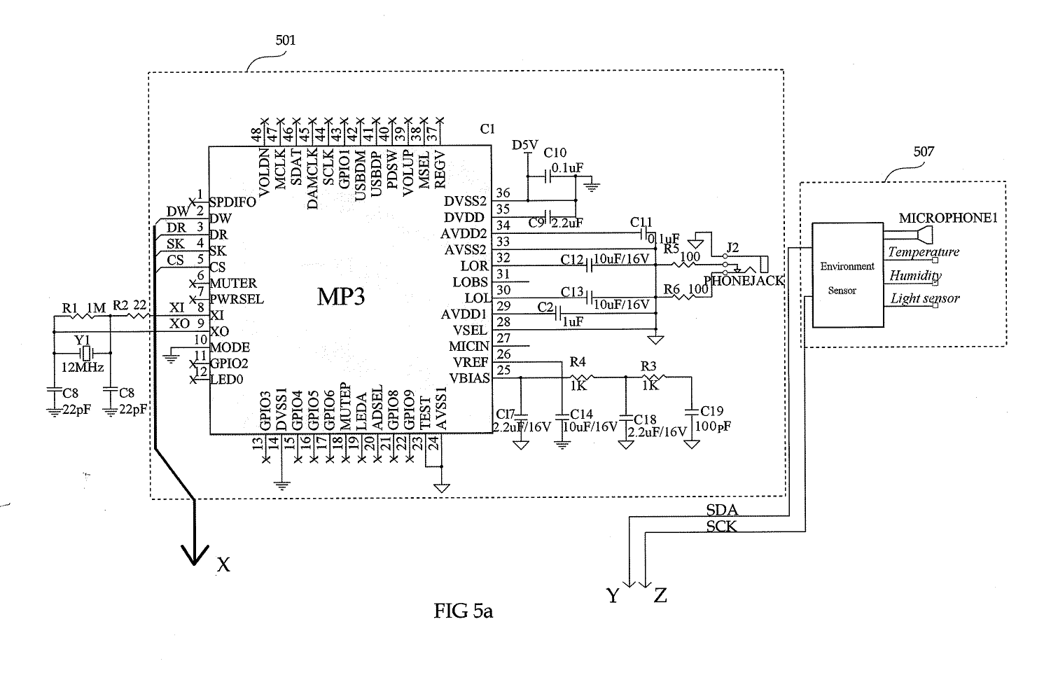

FIG. 3 illustrated a block diagram of a relaxation system according to a third embodiment of the present invention. The relaxation system comprises a sound playing unit 301, a light image emitting unit 302, a smell releasing unit 303, a processing unit 304, a far infrared ray emitting unit 305, a fingerprint scanning unit 306, and a storage unit 307, in which the sound playing unit 301, the light image emitting unit 302, the smell releasing unit 303, the far infrared ray emitting unit 305, the fingerprint scanning unit 306, and the storage unit 307 are connected to the processing unit 304 respectively. The structures of the above units are similar to those in the second embodiment, thus the details of which would not be repeated here.

The present embodiment further comprises an environmental sensor unit 310, which may include a microphone 311, a blood pressure meter 312 and a heartbeat detector 313.

The environmental sensor unit 310 is used to detect a variety of environmental information and transmit the same to the processing unit 304. The processing unit 304 roughly evaluates the stress level of the user according to the detected environmental information, and then controls the corresponding stress relief units to relieve such stress. The environmental information mentioned in this embodiment includes the information on the body of the user, as well as other information irrelevant to the body of the user. The variety of environmental information may incur stress to human being, while the variety of physiological information on human body in turn reflects the stress level being borne. Therefore, some precautions may be taken to prevent the stress of human being from increasing by detecting such kind of environmental information, which could also relieve the stress of human being to some extent.

The microphone 311 is used to detect noise signal in the environment. The noise signal is an important factor to incur stress. The higher the level of the noise signal is, the heavier the incurred stress is. Thus, the detection of the noise signal could perfectly reflect the stress level which is or will be borne by human body.

As a sample of the present invention, after receiving the noise signal detected by the microphone 311, the processing unit 304 will evaluate the stress level might be incurred to the user by the noise signal, and then control one or more units among the sound playing unit 301, the light image emitting unit 302, the smell releasing unit 303 and the far infrared ray emitting unit 305 to relieve such stress.

As another sample of the present invention, after receiving the noise signal detected by the microphone 311, the processing unit 304 will control the sound playing unit 301 to play a sound opposite to this noise signal. Then, the sound and the noise signal will be harmonized with each other to lower the effect of the noise signal to the user.

The blood pressure meter 312 is used to measure the blood pressure of the user. When an individual is in nervous emotional state, his blood pressure would rise accordingly. Therefore, the stress level of human body could be reflected by the blood pressure value. The processing unit 304 may control the relaxation system to take corresponding measures to relieve the stress according to the blood pressure value detected by the blood pressure meter 312.

The heartbeat detector 313 is used to measure the heartbeat rate of human being. As we know, when an individual is under pressure, the nervous emotion would raise the heartbeat rate thereof. Therefore, the stress level of human body could be reflected by the heartbeat rate thereof.

As an embodiment of the present invention, the environmental sensor unit 310 may not only include the above-mentioned microphone 211, the blood pressure meter 312 and the heartbeat detector 313, but also other sensors for collecting various kinds of information.

For example:
Optical sensor, being used to detect light intensity in the environment. Different color components of ambient light caused by different kinds of natural and/or artificial light sources or filtered light may result in different levels of stress in human body.
Camera, being used to collect the images visible for human being, especially certain identifiable subjects, such as human face, tools and so on. When an individual see an angry face, a sword or a gun, his stress level would rise.
Temperature gauge, being used to measure the environmental temperature. The abnormal high or low environmental temperature will incur different levels of stress to human body. To the contrary, a suitable environmental temperature could relieve stress of human being and make one comfortable.

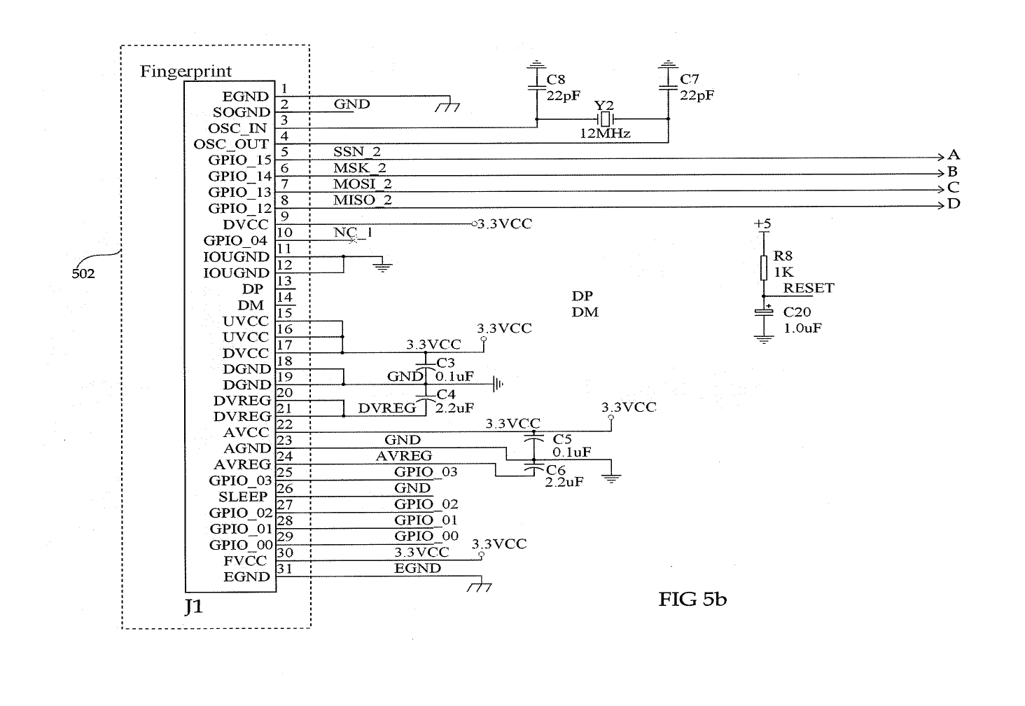

As an embodiment of the present invention, the environmental sensor unit 310 could be directly connected to one or more units among the sound playing unit 301, the light image emitting unit 302, the smell releasing unit 303, and the far infrared ray emitting unit 305. Thus, when the environmental sensor unit 310 collects corresponding environmental information, the above units could be actuated directly to relieve the stress of human body. As shown in FIG. 4, the environmental sensor unit 310 is directly connected to the sound playing unit 301 and the far infrared ray emitting unit 305. More particularly, the microphone 311 and the blood pressure meter 312 are connect to the sound playing unit 301 respectively, while the heartbeat detector 313 is connected to the far infrared ray emitting unit 305 separately. Then, when the sound playing unit receive signal from the microphone 311 and/or blood pressure meter 312, it will play corresponding sound to relieve the stress of the user. Similarly, when the far infrared ray emitting unit 305 receives signal from the heartbeat detector 313, it will also emit far infrared rays to relieve the stress of the user.

As an embodiment of the present invention, at least two units among the sound playing unit 301, the light image emitting unit 302, the smell releasing unit 303 and the far infrared ray emitting unit 305 may operate synchronously. The manner of synchronization between those units is similar to that in the second embodiment, and the details of which would not be repeated here.

As an embodiment of the present invention, the relaxation system further comprises a massage unit. The massage unit could be a mechanical massage unit for offering the user a mechanical massage service for stress relief. The massage unit could not only be connected to the processing unit 304, but also to either unit among the sound playing unit 301, the light image emitting unit 302, the smell releasing unit 303, the far infrared ray emitting unit 305, and the environmental sensor unit 310.

On basis of the first and second embodiment, an environmental sensor unit is added to the present embodiment, therefore the present embodiment of the relaxation system could intelligently perform corresponding operation for stress relief in accordance with the environment. Therefore, the incidence of various psychological and physiological disorders resulted from stress of human body could be reduced.

### THE FOURTH EMBODIMENT

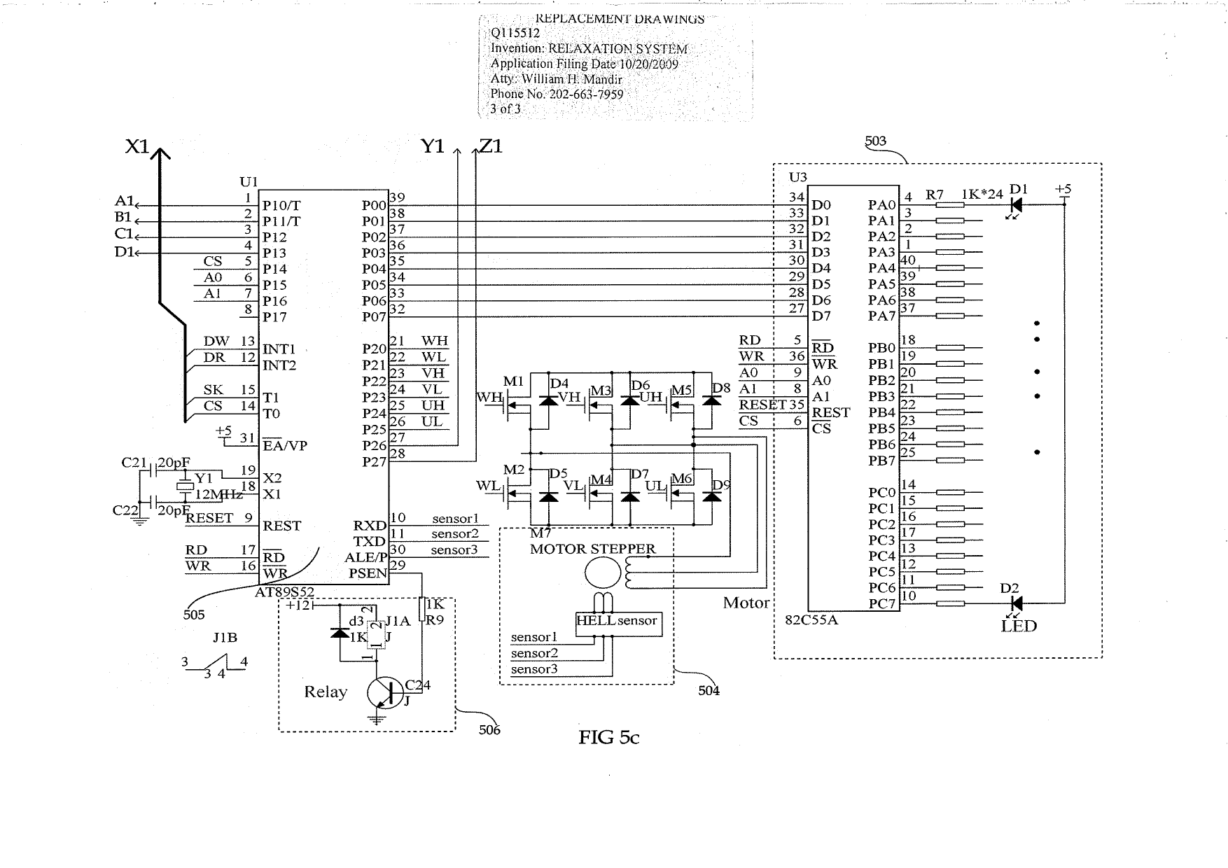

A detailed circuit diagram of the relaxation system is used to illustrate the present embodiment. FIG. 5 illustrates a circuit diagram of the relaxation system according to the fourth embodiment of the present invention. As shown in the figure, the circuit of the relaxation system comprises a MP3 playing unit 501, a fingerprint scanning unit 502, a light image emitting unit 503, a massage unit 504, a CPU 505, and an environmental sensor unit 507.

In the present embodiment, the CPU 505 may be an AT89S52 microcontroller, which is a powerful 8-bit CMOS microcontroller with low power consumption. As shown in the figure, the MP3 playing unit 501, the fingerprint scanning unit 502, the light image emitting unit 503, the massage unit 504, and the environmental sensor unit 507 are all connected to the CPU 505. In particular, the MP3 playing unit 501 is connected to the CPU 505 through interrupt input ports INT0, INT1 and timer ports T0, T1 of the latter. The fingerprint scanning unit 502 is connected to the CPU 505 through I/O ports P10-P13 of the latter. The light image emitting unit 503 is connected to the CPU 505 through I/O ports P00-P07 of the latter. The massage unit 504 is connected to WH port and WL port of the CPU 505 through a step motor. The environmental sensor unit 507 is connected to the CPU 505 through I/O ports P27-P28 of the latter. In addition, to drive the CPU 505, a relay 506 is incorporated in the circuit, which is used to supply power to the CPU 505.

As an embodiment of the present invention, the light image emitting unit 503 is provided with an 82C55A chip, 24 I/O pins of which are connected to light emitting diodes, i.e. LEDs respectively, and digital input ports D0-D7 of which are connected to the I/O ports P10-P13 of the CPU 505. Therefore, the CPU 505 could control each of the LEDs to emit light through the 82C55A chip, to display corresponding light image for stress relief.

As an embodiment of the present invention, the environmental sensor unit 507 includes a plurality of sensors. In the present embodiment, it includes a microphone, a temperature sensor, a humidity sensor and a light sensor. Of course, other sensors could also be used as the environmental sensor unit of the present embodiment, such as a blood pressure meter, heartbeat detector, etc.

As an embodiment of the present invention, the massage unit 504 could a mechanical massage unit which could further comprise a sensor device for receiving signal from various sensors. For instance, the massage unit 504 could receive signal from the environmental sensor unit 507 and then offer the user a massage for fatigue relief according the received signal. Of course, the massage unit 504 could also receive other signal received from other units, such as the sound playing unit 501, the light image emitting unit 503, etc. of the present relaxation system to perform a massage.

As an embodiment of the present invention, the MP3 playing unit 501 and fingerprint scanning unit 502 could also be provided with a chip know in the prior art, the details of which would not be repeated here.

Although the description of the present invention is made with reference to the preferred embodiments, the present invention is not limited to these embodiments. Various modifications and changes can be made to the invention by those skilled in the art without departing from the spirit and scopes of the present invention.

What is claimed is:

1. A relaxation system comprising a processing unit, a sound playing unit, a light image emitting unit, a smell releasing unit and a fingerprint scanning unit, in which the sound playing unit, the light image emitting unit, the smell releasing unit and the fingerprint scanning unit are connected to the processing unit respectively, the sound playing unit is used to play sounds, the light image emitting unit is used to emit light, the smell releasing unit is used to release smell, the fingerprint scanning unit is used to collect fingerprint information and transmit the collected fingerprint information to the processing unit, the processing unit is used to control the operation of the sound playing unit, the light image emitting unit and the smell releasing unit and perform a fingerprint identification according to the fingerprint information collected by the fingerprint scanning unit,
wherein the system further comprises an environmental sensor unit, and the environmental sensor unit is connected to the processing unit and is used to collect environmental information for the processing unit to enable the processing unit to control the operation of the sound playing unit, the light image emitting unit and the smell releasing unit according to the collected environmental information, and
wherein the environmental sensor unit comprises a microphone for detecting noise signal in the environment.

2. The relaxation system according to claim 1, wherein the smell releasing unit comprises at least one fragrance carrier, and a microprocessor for selecting one of the at least one fragrance carrier and controlling the operation of the selected fragrance carrier.

3. The relaxation system according to claim 2, wherein each of the at least one fragrance carrier comprises a container for containing a kind of fragrance liquid, a cylinder extending upwards from an opening provided with the container and comprising a through hole, a absorption element provided within the through hole of the cylinder for conveying the fragrance liquid contained in the container, an electrical heating element attached to the cylinder and connected to a power source to be supplied with power.

4. The relaxation system according to claim 3, wherein the cylinder is made of metal or other thermal conductive materials.

5. The relaxation system according to claim 3, wherein one or more of the at least one fragrance carrier are associated with an anion generator to enable to produce anion during the operation of the former.

6. The relaxation system according to claim 1, further comprising a storage unit for storing sounds and fingerprint information, wherein the storage unit is connected to the processing unit, and the processing unit is used to determine whether fingerprint information collected by the fingerprint scanning unit is authorized in accordance with the fingerprint information stored in the storage unit and control the sound playing unit to play the sounds stored in the storage unit.

7. The relaxation system according to claim 1, further comprising a far infrared ray emitting unit for emitting far infrared rays, wherein the far infrared ray emitting unit is connected to the processing unit, and the processing unit is used to control the far infrared ray emitting unit to emit far infrared rays.

8. The relaxation system according to claim 1, wherein the environmental sensor unit comprises a heartbeat detector for detecting heartbeat rate of a user.

9. The relaxation system according to claim 1, wherein the environmental sensor unit comprises a blood pressure meter for measuring blood pressure of a user.

10. The relaxation system according to claim 1, wherein the light image emitting unit is a light source capable of emitting light in a variety of colors, and is connected to one or more units among the sound playing unit, the smell releasing unit and the environmental sensor unit to enable to operate synchronously.

11. The relaxation system according to claim 1, further comprising a massage unit for offering a massage to a user.
